(19) United States
(12) Patent Application Publication
(10) Pub. No.: US 2026/0053406 A1
(43) Pub. Date: Feb. 26, 2026

(54) SYSTEM AND METHOD OF PREDICTING DISPOSITION OF A MENTAL DISORDER OF A SUBJECT

(71) Applicants: CARMEL HAIFA UNIVERSITY ECONOMIC CORPORATION LTD., Haifa (IL); SALK INSTITUTE FOR BIOLOGICAL STUDIES, La Jolla, CA (US); NOVA SCOTIA HEALTH AUTHORITY, Halifax (CA)

(72) Inventors: Shani STERN, Haifa (IL); Fred GAGE, La Jolla, CA (US); Martin ALDA, Halifax (CA); Liron MIZRAHI, Kiryat Bialik (IL)

(21) Appl. No.: 19/103,764

(22) PCT Filed: Aug. 14, 2023
(86) PCT No.: PCT/IL2023/050856

§ 371 Date: Feb. 13, 2025

Related U.S. Application Data
(60) Provisional application No. 63/397,857, filed on Aug. 14, 2022.
(60) Provisional application No. 63/460,414, filed on Apr. 19, 2023.

(51) Int. Cl. A61B 5/16; G16B 25/10; G16B 40/20; G16H 10/60

(52) U.S. Cl. CPC A61B 5/165; G16B 25/10; G16B 40/20; G16H 10/60

(57) ABSTRACT

A system and method of predicting disposition of a mental disorder of a subject may include obtaining a Lymphoblastoid Cell Line (LCL) assay of the subject; calculating a gene expression profile of the subject based on the LCL assay, wherein said gene expression profile comprises a plurality of gene expression levels, each representing quantity of a respective RNA molecule in the LCL assay; providing a first machine-learning (ML) based model, pretrained to predict disposition of a mental disorder based on gene expression profile data; and applying the first ML-based model on the gene expression profile of the subject, to predict disposition of the mental disorder in the subject.

### CROSS-REFERENCE TO RELATED APPLICATIONS

This application claims the benefit of U.S. patent application Ser. No. 63/397,857, filed Aug. 14, 2022, and titled: “PATIENT SPECIFIC EXPRESSION PATTERN OF CERTAIN IMMUNOGLOBULIN GENES PREDICT LITHIUM RESPONSE IN PATIENTS AFFLICTED WITH BIPOLAR DISORDER”, and U.S. patent application Ser. No. 63/460,414, filed Apr. 19, 2023 and titled: “USING INFORMATION THEORY AND MACHINE LEARNING TO PREDICT LITHIUM RESPONSE IN BIPOLAR DISORDER PATIENTS”, which are both hereby incorporated by reference in their entirety.

### FIELD OF THE INVENTION

The present invention relates generally to the field of computer-assisted healthcare systems. More specifically, the present invention relates to prediction of disposition to a mental disorder.

### BACKGROUND OF THE INVENTION

Bipolar Disorder (BD) is a mental disorder characterized by periods of depression and periods of abnormally elevated mood that may each last from days to weeks. Mania is a state of extremely elevated energy levels that may be associated with psychosis. During mania, an individual may feel abnormally energetic, happy or irritable, and may often make impulsive decisions with little regard for the consequences. During periods of depression, the individual may experience a negative outlook on life, and may be prone to suicide or self-harm.

Accurate prediction of an individual's disposition to bipolar disorder and their potential responsiveness to specific treatments is therefore a crucial step towards early intervention and improved patient outcomes.

### SUMMARY OF THE INVENTION

Traditionally, diagnosing BD and determining suitable treatment strategies has relied heavily on clinical observation, subjective assessments, and the expertise of mental health professionals. However, these approaches often suffer from variability, subjectivity, and a reliance on self-reporting, which can hinder the timely and accurate identification of the disorder, leading to delays in appropriate treatment initiation.

Advances in the field of artificial intelligence (AI) have shown promise in transforming healthcare by leveraging large datasets, complex algorithms, and machine learning techniques to extract meaningful patterns and insights from diverse sets of data.

As elaborated herein, embodiments of the invention may employ machine learning (ML) based techniques and models to analyze data originating from a range sources, such as electronic health records and genetic information, to identify subtle indicators that might not be apparent through conventional clinical assessments alone.

Specifically, embodiments of the invention may allow prediction of disposition to BD, to identify individuals who are at a higher risk of developing the disorder, and enable targeted interventions, personalized treatment plans, and the implementation of preventative strategies.

Additionally, embodiments of the invention may predict an individual's potential responsiveness to specific treatments, such as Lithium, a commonly used mood stabilizer, thereby significantly enhancing treatment efficacy, and reducing the trial-and-error approach that is often associated with psychiatric medication. Embodiments of the invention may include a method of predicting disposition of a mental disorder of a subject or patient by at least one processor.

Embodiments of the method may include obtaining a Lymphoblastoid Cell Line (LCL) assay of the subject; calculating a gene expression profile of the subject based on the LCL assay, wherein said gene expression profile may include a plurality of gene expression levels, each representing quantity of a respective RNA molecule in the LCL assay; providing a first machine-learning (ML) based model, pretrained to predict disposition of a mental disorder based, at least in part, on gene expression profile data; and applying the first ML-based model on the gene expression profile of the subject, to predict disposition of the mental disorder in the subject.

According to some embodiments, the mental disorder may include, for example a Bipolar Disorder (BD), a manic condition, and a condition of depression.

According to some embodiments, the processor may identify, in the plurality of RNA molecules, a first subset of RNA molecules as differentially expressed between a first group of subjects, having the mental disorder, and a second, control group of subjects, beyond a predefined threshold. The at least one processor may apply the first ML-based model on the gene expression profile by applying the first ML-based model on the (e.g., only on the) gene expression levels of the first subset of RNA molecules.

According to some embodiments, the first subset of RNA molecules may respectively correspond to a group of genes that may include UBAP1L, OAZ3, RPL7P6, MTND5P15 and IGSF9T.

Additionally, or alternatively, the first subset of RNA molecules may respectively correspond to a group of genes that may include, for example, MYO1H, RPL29P33, OAZ3, RPL7P6, PPP1R3F, IGSF9, MTND5P15, UBAP1L, NEK10, SRC, PCDHGB7, SNORA20, DCBLD2, MRM2, TSACC, PPFIA1, ZC3H14, CHRM5, FRG1CP, and ZNF346.

According to some embodiments, the processor may obtain clinical data representing historical manifestations of the mental disorder in the subject; and apply the first ML-based model on the clinical data, in addition to the gene expression profile of the subject, to predict disposition of the mental disorder in the subject.

Embodiments of the invention may provide a second ML based model, pretrained to predict responsiveness to a treatment associated with the mental disorder based, at least in part, on gene expression profile data. In such embodiments, the at least one processor may apply the second ML-based model on the gene expression profile of the subject, to predict responsiveness of the subject to the treatment.

According to some embodiments, the mental disorder may include a Bipolar Disorder (BD), a manic condition, and a condition of depression, and the treatment may include intake of Lithium.

According to some embodiments, the at least one processor may identify, in the plurality of RNA molecules, a second subset of RNA molecules as differentially expressed between a first group of subjects, responsive to the treatment, and a second group of subjects, not responsive to the treatment, beyond a predefined threshold. The at least one processor may apply the second ML-based model on the gene expression profile by applying the second ML-based model on the gene expression levels of the second subset of RNA molecules.

According to some embodiments, the second subset of RNA molecules may respectively correspond to a group of genes that may include, for example EEF1A1P34, NRIP2, GPR63, ADAM20P1, GLRA2, HCP5B and TERB1.

Additionally, or alternatively, the second subset of RNA molecules may respectively correspond to a group of genes that may include, for example, EEF1A1P34, NRIP2, GPR63, ADAM20P1, GLRA2, HCP5B, TERB1,SCAT2, NUSAP1, ZNF93, C16orf96, SNORA20, GPX2, IGHV5-51, CRYZ, WDR5-DT, IGLV1-47 and IGHV4-80.

Additionally, or alternatively, the at least one processor may obtain clinical data representing historical manifestations of the mental disorder in the subject. The at least one processor may subsequently apply the second ML-based model on the clinical data, in addition to the gene expression profile of the subject, to predict responsiveness of the subject to the treatment.

Embodiments of the invention may include A system for predicting disposition of a mental disorder of a subject. Embodiments of the system may include: a non-transitory memory device, wherein modules of instruction code are stored, and at least one processor associated with the memory device, and configured to execute the modules of instruction code. Upon execution of said modules of instruction code, the at least one processor may be configured to: obtain a Lymphoblastoid Cell Line (LCL) assay of the subject; calculate a gene expression profile of the subject based on the LCL assay, wherein said gene expression profile may include a plurality of gene expression levels, each representing quantity of a respective RNA molecule in the LCL assay; provide a first machine-learning (ML) based model, pretrained to predict disposition of a mental disorder based, at least in part, on gene expression profile data; and apply the first ML-based model on the gene expression profile of the subject, to predict disposition of the mental disorder in the subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for prediction of disposition to a mental disorder, according to some embodiments;

FIG. 2 is a block diagram, depicting a system for prediction of disposition to a mental disorder, according to some embodiments;

FIG. 3 is a diagram of Differentially Expressed Genes (DEGs), showing a comparison between expression of genes in Lymphoblast Cell Lines (LCLs) of BD patients (top section) and expression of genes in LCLs of control (non-BD) subjects, according to some embodiments;

FIG. 4 is a diagram showing gene expression levels of genes (or corresponding RNA molecules). Each panel of FIG. 4 shows expression of a specific gene in a BD-diagnosed group of patients (“BD”) and in a control group of subjects (“CTRL”), on logarithmic scale, according to some embodiments;

FIG. 5 is a graph that presents Receiver Operating Characteristic (ROC) curves, illustrating the diagnostic ability of different types of architectures of a machine learning model as a binary classifier, to distinguish between BD and non-BD subjects, based on gene expression levels, according to some embodiments;

FIG. 6 is a diagram of DEGs, showing a comparison between expression of genes (e.g., levels of sequenced RNA molecules) in LCLs of BD patients, who are responsive to Lithium treatment (LR) (top section) and expression of genes in LCLs of BD patients who are not responsive to Lithium treatment (NR) (bottom section), according to some embodiments;

FIG. 7 is a diagram showing gene expression levels of genes (or corresponding RNA molecules) within a subset of selected genes. Each panel of FIG. 7 shows expression of a specific gene in Lithium non-responsive group of patients (‘0’) and in a Lithium responsive group of patients (‘1’), on logarithmic scale.

FIG. 8 is a graphs which presents ROC curves, illustrating the diagnostic ability of different types of architectures of a machine learning model as a binary classifier, to distinguish between Lithium responsive (LR) and Lithium non-responsive (NR) patients, based on gene expression levels, according to some embodiments of the invention;

FIG. 9 is a flow diagram depicting a method of predicting disposition of a mental disorder in a subject, by at least one processor, according to some embodiments of the invention; and

FIGS. 10A and 10B, are flow diagrams depicting a process of training ML models, by a training module, so as to predict (e.g., produce a prediction of) a condition of a subject (e.g., BD disposition), and/or predict (e.g., produce a prediction 140P of) the patient's responsivity to treatment, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

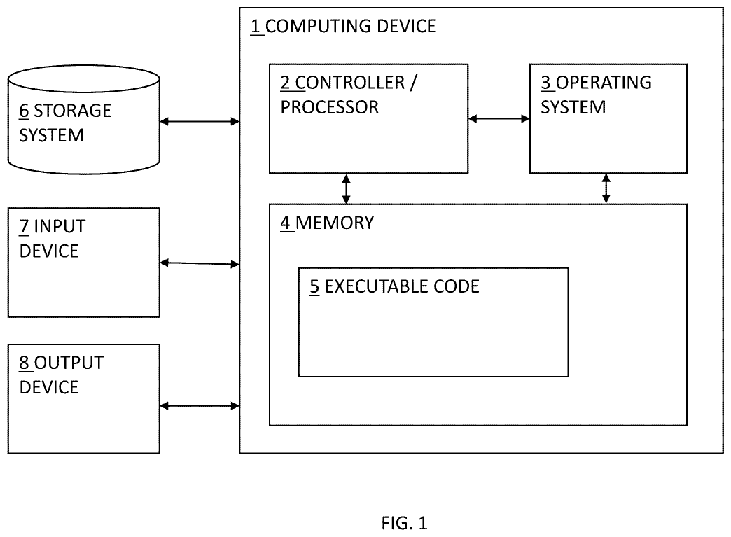

Reference is now made to FIG. 1, which is a block diagram depicting a computing device, which may be included within an embodiment of a system for prediction of disposition to a mental disorder, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may predict disposition to a mental disorder as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a specific subject or patient may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

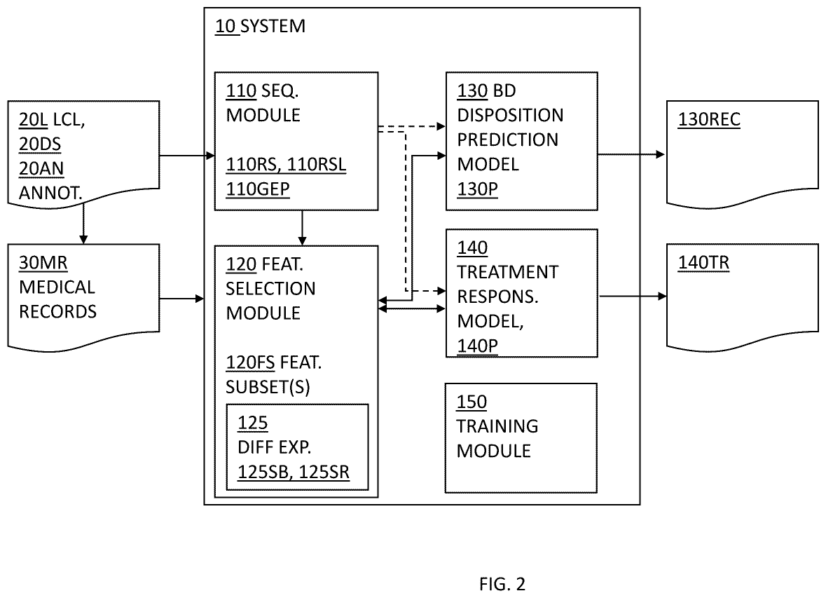

Reference is now made to FIG. 2, which depicts a system 10 for prediction of disposition to a mental disorder, according to some embodiments.

According to some embodiments of the invention, system 10 may be implemented as a software module, a hardware module, or any combination thereof. For example, system 10 may be or may include a computing device such as element 1 of FIG. 1, and may be adapted to execute one or more modules of executable code (e.g., element 5 of FIG. 1) to predict of disposition to a mental disorder, as further described herein.

As shown in FIG. 2, arrows may represent flow of one or more data elements to and from system 10 and/or among modules or elements of system 10. Some arrows have been omitted in FIG. 2 for the purpose of clarity.

As shown in FIG. 2, system 10 may obtain a Lymphoblastoid Cell Line (LCL) assay 20L of a subject or patient of interest. As known in the art, LCLs may be obtained by infecting Peripheral Blood Mononuclear Cells (PBMCs) with the Epstein-Barr virus (EBV). By doing so, EBV may immortalize human B cells in vitro, enabling them to proliferate with an average population doubling time of approximately 24 hours.

According to some embodiments, system 10 may include (as depicted in FIG. 2), or may be associated with a sequencing device or module 110, also referred to herein as “sequencer 110” for short.

Sequencer 110 may be configured to produce Ribonucleic Acid (RNA) sequences 110PS from a biological sample, as known in the art. In some embodiments of the invention, sequencer 110 may produce, or calculate a gene expression profile 110GEP of the subject or patient, based on the LCL assay 20L. The gene expression profile may also be referred to herein as a transcriptome 110GEP. Gene expression profile 110GEP may be a data element (e.g., a table) that includes a plurality of gene expression levels 110RSL, each representing quantity of a respective, sequenced RNA molecule 110RS in LCL assay 20L.

As known in the art, RNA molecules may be obtained via a biological process of transcription of corresponding genes. In other words, sequenced RNA molecules 110RS may be regarded as an expression of corresponding genes. Therefore, the terms RNA molecules 110RS and expressed genes 110RS may be used interchangeably, according to context.

As shown in FIG. 2, system 10 may include one or more machine-learning (ML) based models 130, 140, and a training module 150. According to some embodiments, a first ML model 130 may be pretrained to predict disposition of a mental disorder in a patient based, at least in part, on gene expression profile data 110GEP.

For example, during a training period, system 10 may receive a training dataset 20DS that may include a plurality of LCLs 20L. Additionally, or alternatively, system 10 may receive (e.g., via input 7 of FIG. 1) a training dataset that may include gene expression profiles 110GEP data elements corresponding to respective LCL assays 20L.

The training dataset 20DS may be labeled, or annotated, in a sense that one or more (e.g., each) gene expression profiles 110GEP of the training dataset 20DS, and/or one or more (e.g., each) data element or LCLs 20L of the training dataset 20DS may be attributed a respective annotation data element 20AN.

Annotation data element 20AN may, for example include an indication regarding the disposition of a respective patient to suffer from BD. System 10 may thus utilize training module 150 to train ML model 130, so as to predict disposition of a mental disorder based, at least in part, on gene expression profile data 110GEP, using the annotation data elements 20AN as supervisory information.

During an inference period, which may be subsequent to, or intermittent with the training period, system 10 may infer, or apply ML model 130 on the gene expression profile data element 110GEP of a specific, target subject. System 10 may thus employ ML model 130 to predict disposition of the mental disorder of interest.

In other words, ML model 130 may be used by one or more processors (e.g., processor 2 of FIG. 1) to identify disposition, e.g., a current or future expected onset of a mental disorder such as BD, a manic condition, a condition of depression, and the like. Additionally, or alternatively, ML model 140 may emit a recommendation 130REC, or notification that may include, for example, a diagnosis for the specific patient, indicating their disposition to be, develop BD.

According to some embodiments, system 10 may include a feature selection module 120, adapted to select specific features, e.g., levels 110RSL of specific RNA sequences 110RS as input for ML model 130.

For example, feature selection module 120 may include a differential expression module 125, configured to identify, in the plurality of sequenced RNA molecules 110RS, a subset 125SB of RNA molecules that are differentially expressed, beyond a predefined threshold, between a first group of subjects having the mental disorder (e.g., BD), and a second, control group of subjects, which may not have the mental disorder.

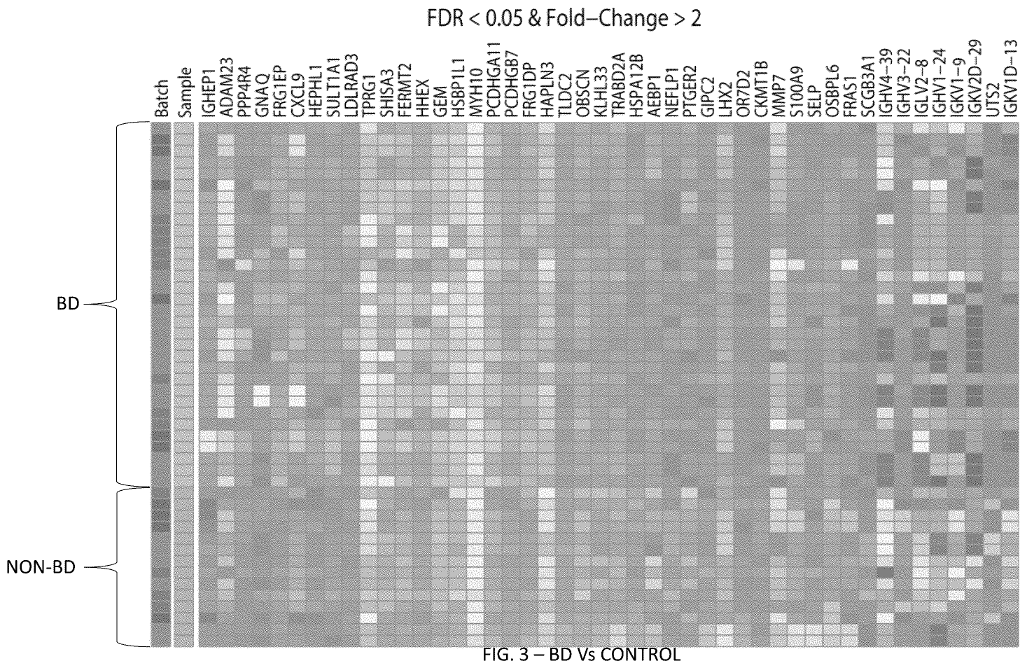

Reference is also made to FIG. 3, which is a diagram of Differentially Expressed Genes (DEGs), showing a comparison between expression of genes (e.g., RNA levels 110RSL) in Lymphoblast Cell Lines (LCLs) 20L of BD patients (top section) and expression of genes 110RSL in LCLs 20L of control (non-BD) subjects (bottom section).

In FIG. 3, each gene or sequenced RNA molecule 110RS of subset 125SB is presented in a dedicated column, where each row represents expression in a specific subject or patient of a cohort of subjects. The expression levels 110RSL of each gene or sequenced RNA molecule 110RS of subset 125SB is represented by a dedicated hue, or brightness scale (e.g., where a dark hue represents a high expression level 110RSL, and a light hue represents a low expression level 110RSL).

As known in the art, a gene may be regarded as differentially expressed if an observed difference or change in read counts or expression levels 110RSL between two experimental conditions is statistically significant, beyond a predefined threshold. In the case of FIG. 3, such a difference in expression levels 110RSL of genes of subset 125SB is visually detectable by the hue and intensity of pixels in the table.

According to some embodiments, system 10 may apply ML-based model 130 on (e.g., only on) the gene expression levels 110RSL of the subset 125SB of RNA molecules of gene expression profile 110GEP.

In other words, system 10 may omit or filter-out expression levels 110RSL of genes (e.g., of corresponding RNA sequences 110RS) that are not differentially expressed between BD patients and the control group, as input for ML model 130, and infer ML model 130 only on levels 110RSL of subset 125SB to predict disposition of the subject to a mental disorder such as BD.

Additionally, or alternatively, feature selection module 120 may apply any appropriate algorithm of feature selection, as known in the art, to extract genes, or corresponding sequences of RNA 110RS, whose gene expression levels 110RSL are most indicative for classification of LCL 20L as pertaining to classification of subjects as BD patients or non-BD patients.

As shown in FIG. 2, system 10 may obtain or receive (e.g., via input device 7 of FIG. 1) clinical data, or medical records 30MR representing historical manifestations of the mental disorder in the subject. According to some embodiments, system 10 may apply ML-based model 130 on the clinical data 30MR, in addition to data of gene expression profile 110GEP (or subset 125SB) of the subject, to predict disposition of the mental disorder (e.g., BD) in the subject.

In other words, training dataset 20DS may further include information representing medical records 30MR, and respective annotations 20AN of specific patients, indicating their medical condition, and system 10 may utilize training module 150 to train ML model 130 further based on this 30MR data, using annotations 20AN as supervisory information, to predict disposition of a mental disorder (e.g., BD) in target subjects.

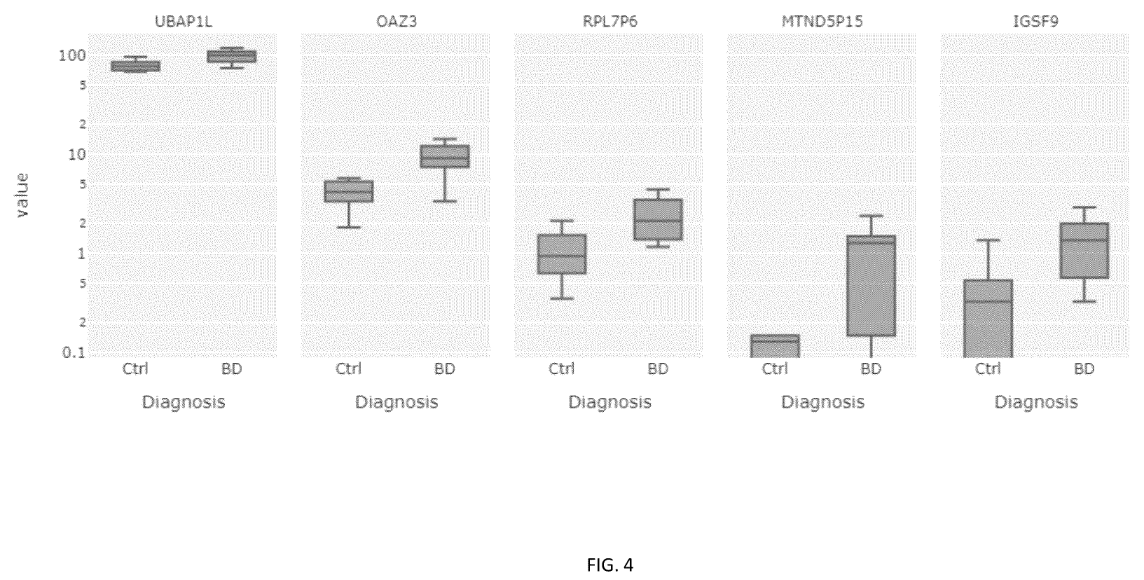

Reference is now made to FIG. 4 which is a diagram showing gene expression levels 110RSL of genes (or corresponding RNA molecules) of subset 125SB. Each panel of FIG. 4 shows expression of a specific gene in a BD-diagnosed group of patients (“BD”) and in a control group of subjects (“CTRL”), on logarithmic scale. As shown in FIG. 4, a subset 125SB of RNA molecules that are differentially expressed between subjects having the mental disorder (e.g., BD), and a second, control group of subjects who do not have the mental disorder, may respectively correspond to a group of genes selected from: UBAP1L, OAZ3, RPL7P6, MTND5P15 and IGSF9T.

It may be appreciated that the inventors have experimentally identified additional genes or corresponding RNA molecule sequences 110RS that are also indicative of classification of subjects as BD or non-BD subjects. Accordingly, subset 125SB of sequenced RNA molecules 110RS may respectively correspond to a group of genes further selected from MYO1H, RPL29P33, OAZ3, RPL7P6, PPP1R3F, IGSF9, MTND5P15, UBAP1L, NEK10, SRC, PCDHGB7, SNORA20, DCBLD2, MRM2, TSACC, PPFIA1, ZC3H14, CHRM5, FRG1CP, and ZNF346.

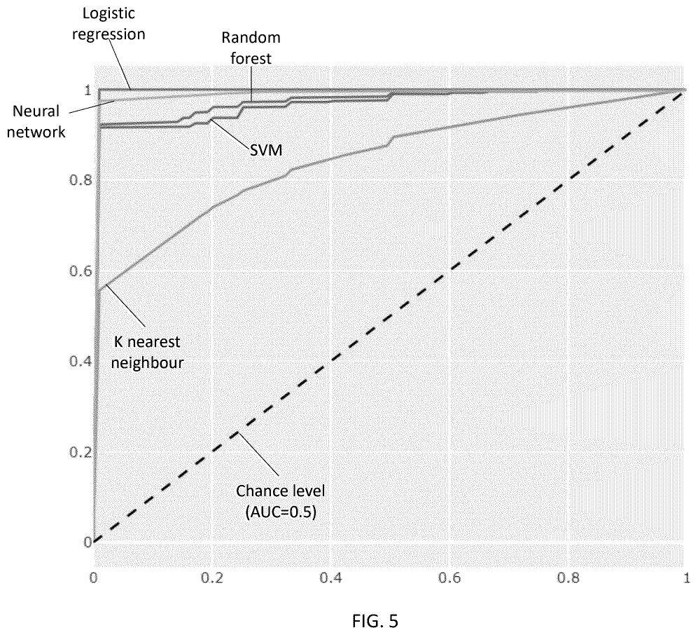

Reference is now made to FIG. 5 which presents Receiver Operating Characteristic (ROC) curves, illustrating the diagnostic ability of different types of architectures of ML model 130 as a binary classifier, to distinguish between BD and non-BD subjects, based on gene expression levels 110RSL of subset 125SB. As shown in FIG. 5, the inventors have examined the ROC curves of various ML model architectures, including a Logistic Regression model, a Neural Network (NN) model, a Random Forest model, a Support Vector Machine (SVM) classifier and a K-nearest neighbour model. Experimental results have shown the best Area Under Curve (AUC) for the Logistic Regression model, and the worst AUC to be that of the K-nearest neighbour model. Accordingly, ML model 130 may be selected to be a Logistic Regression model.

As shown in FIG. 2, system 10 may include, or provide a second ML model 140 that may be pretrained to predict responsiveness of a patient to treatment associated with the mental disorder (e.g., BD) based, at least in part, on gene expression profile data 110GEP.

For example, during a training period, system 10 may receive a training dataset 20DS that may include a plurality of LCLs 20L. Additionally, or alternatively, system 10 may receive (e.g., via input 7 of FIG. 1) a training dataset that may include gene expression profile 110GEP data elements corresponding to respective LCL assays 20L (e.g., of specific subjects).

The training dataset 20DS may be labeled, or annotated, in a sense that one or more (e.g., each) gene expression profiles 110GEP of the training dataset 20DS, and/or one or more (e.g., each) data element or LCLs 20L of the training dataset 20DS may be attributed a respective annotation data element 20AN.

Annotation data element 20AN may, for example, include an indication regarding the responsiveness of a respective patient to a specific treatment (e.g., administering Lithium). Annotation data element 20AN may present such responsiveness levels as a numerical value, that may be binary (e.g., Yes/No) or continuous (e.g., responsiveness on a scale between 1-10).

System 10 may thus utilize training module 150 to train ML model 140, so as to predict responsiveness of a respective patient to treatment based, at least in part, on gene expression profile data 110GEP, while using the annotation data elements 20AN as supervisory information.

During an inference period, which may be subsequent to, or intermittent with the training period, system 10 may infer, or apply ML model 140 on the gene expression profile data element 110GEP of a specific, target subject. System 10 may thus employ ML model 140 to predict responsiveness of the target subject to the treatment.

In other words, ML model 140 may be used by one or more processors (e.g., processor 2 of FIG. 1) to identify or classify an LCL assay 20L as pertaining to a subject who is responsive (LR), or non-responsive (NR) to Lithium treatment. Additionally, or alternatively, ML model 140 may emit a recommendation of treatment 140TR, that may include, for example, a prescription for the specific patient.

According to some embodiments, feature selection module 120 may be adapted to select specific features, e.g., levels 110RSL of specific RNA sequences 110RS as input for ML model 140.

For example, differential expression module 125, may be configured to identify, in the plurality of sequenced RNA molecules 110RS, a subset 125SR of RNA molecules that are differentially expressed, beyond a predefined threshold, between a first group of subjects, who are BD patients, responsive to treatment such as intake of Lithium (also referred to herein as “LR” patients), and a second group of subjects, who are BD patients, and are not responsive to treatment including intake of Lithium (also referred to herein as “NR” patients).

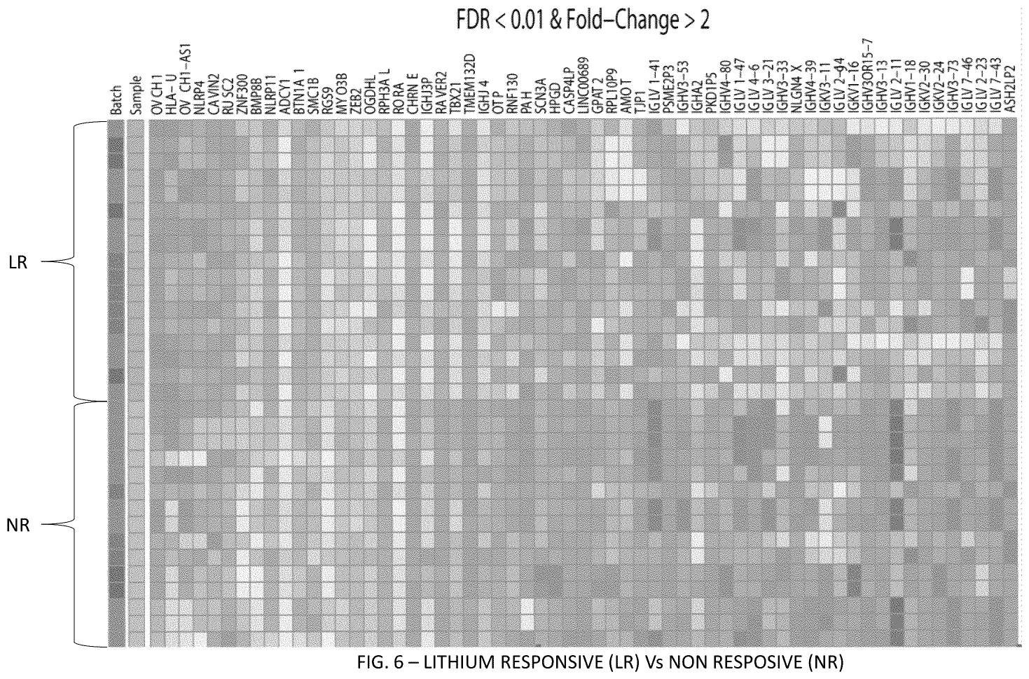

Reference is now made to FIG. 6 which is a diagram of DEGs, showing a comparison between expression of genes (e.g., RNA levels 110RSL) in LCLs 20L of BD patients who are responsive to Lithium treatment (LR) (top section) and expression of genes 110RSL in LCLs of BD patients who are not responsive to Lithium treatment (NR) (bottom section).

In FIG. 6, each gene or sequenced RNA molecule 110RS is presented in a dedicated column, where each row represents expression in a specific subject or patient of a cohort of subjects. The expression levels 110RSL of each gene or sequenced RNA molecule 110RS is represented by a dedicated hue or brightness scale, where a dark hue represents a high expression level 110RSL, and light hue represents a low expression level 110RSL.

The terms “subject” and “patient” may be used herein interchangeably, according to context, to indicating a studied human or animal of interest.

In the case of FIG. 6, the difference in read counts or expression levels 110RSL of genes of subset 125SB between LR patients and NR patients is visually detectable by the hue and intensity of pixels in the table.

According to some embodiments, system 10 may apply ML-based model 140 on (e.g., only on) the gene expression levels 110RSL of the subset 125SR of RNA molecules of gene expression profile 110GEP.

In other words, system 10 may omit or filter-out expression levels 110RSL of genes (e.g., of corresponding RNA sequences 110RS) that are not differentially expressed between LR patients and NR patients, as input for ML model 140, and infer ML model 140 only on levels 110RSL of subset 125SR to predict responsiveness of the subject to the treatment (e.g., Lithium intake).

Additionally, or alternatively, feature selection module 120 may apply any appropriate algorithm of feature selection, as known in the art, to extract genes, or corresponding sequences of RNA 110RS, whose gene expression levels 110RSL are most indicative for classification of LCL 20L as pertaining to BD patients who are LR patients or NR patients.

Additionally, or alternatively, system 10 may apply ML-based model 140 on the clinical data 30MR, in addition to data of gene expression profile 110GEP (or subset 125SR) of the subject, to predict responsiveness to treatment (e.g., intake of Lithium) in the subject.

In other words, training dataset 20DS may further include information representing medical records 30MR, and respective annotations 20AN of specific patients, indicating their medical condition. System 10 may utilize training module 150 to train ML 140 further based on this 30MR data, using annotations 20AN as supervisory information, so as to predict responsiveness to treatment in target subjects.

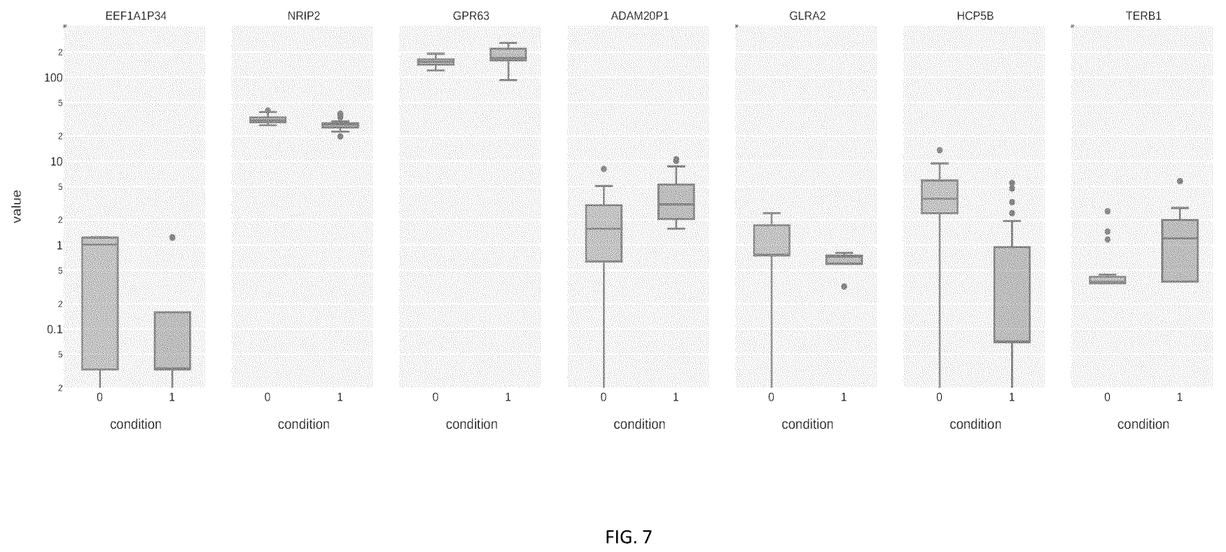

Reference is now made to FIG. 7 which is a diagram showing gene expression levels 110RSL of genes (or corresponding RNA molecules) of subset 125SR. Each panel of FIG. 7 shows expression of a specific gene in an NR group of patients (‘0’) and in an LR group of patients (‘1’), on logarithmic scale. As shown in FIG. 7, a subset 125SR of RNA molecules that are differentially expressed between LR and NR patients may respectively correspond to a group of genes selected from: EEF1A1P34, NRIP2, GPR63, ADAM20P1, GLRA2, HCP5B and TERB1.

It may be appreciated that the inventors have experimentally identified additional genes or corresponding RNA molecule sequences 110RS that are also indicative of classification of subjects as LR or NR patients. Accordingly, subset 125SR of sequenced RNA molecules 110RS may respectively correspond to a group of genes further selected from SCAT2, NUSAP1, ZNF93, C16orf96, SNORA20, GPX2, IGHV5-51, CRYZ, WDR5-DT, IGLV1-47 and IGHV4-80.

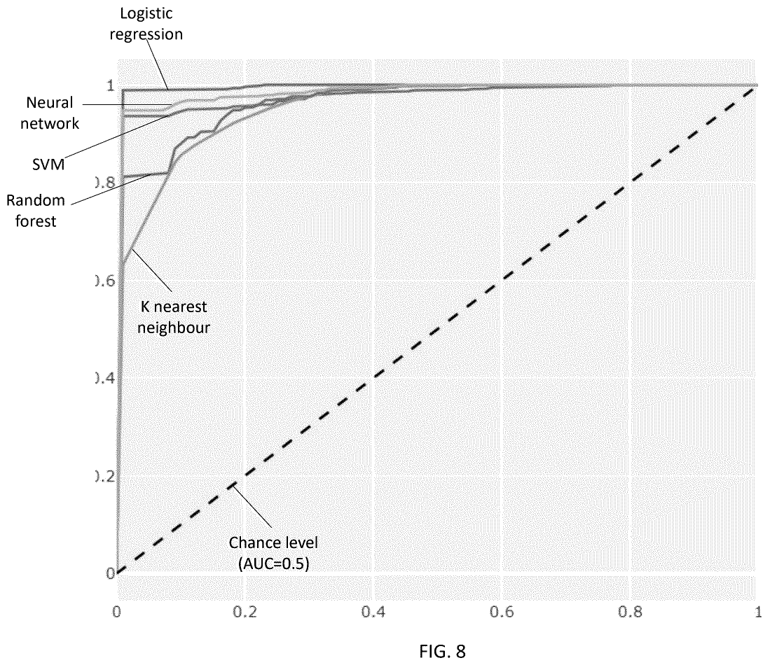

Reference is now made to FIG. 8 which presents Receiver Operating Characteristic (ROC) curves, illustrating the diagnostic ability of different types of architectures of ML model 140 as a binary classifier, to distinguish between LR and NR patients, based on gene expression levels 110RSL of subset 125SR.

As shown in FIG. 8, the inventors have examined the ROC curves of various ML model architectures, including a Logistic Regression model, a Neural Network (NN) model, a Random Forest model, a Support Vector Machine (SVM) classifier and a K-nearest neighbour model. Experimental results have shown the best Area Under Curve (AUC) for the Logistic Regression model, and the worst AUC to be that of the K-nearest neighbour model. Accordingly, ML model 140 may be selected to be a Logistic Regression model.

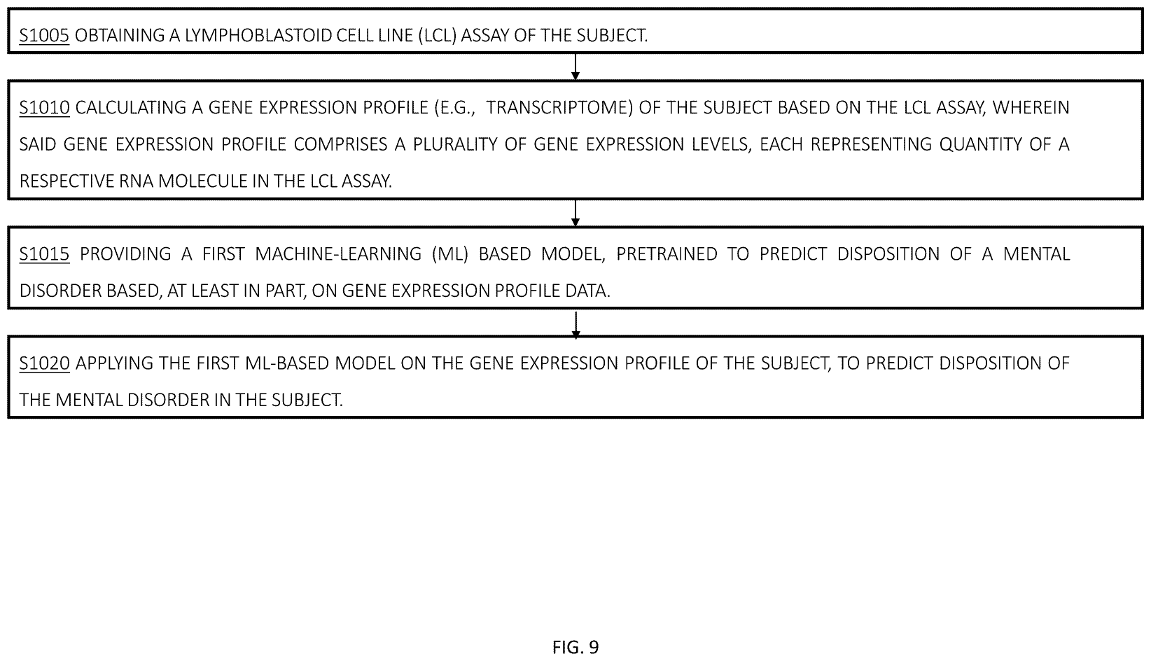

Reference is now made to FIG. 9, which is a flow diagram of a method of predicting disposition of a mental disorder in a subject, by at least one processor (e.g., processor 2 of FIG. 1), according to some embodiments of the invention.

As shown in steps S1005 and S1010, the at least one processor 2 may receive or obtain data representing an LCL assay of the subject.

For example, as elaborated herein (e.g., in relation to FIG. 2), processor 2 may calculate, or may be communicatively connected to a sequencing module 110, adapted to calculate a gene expression profile (e.g., a transcriptome) of the subject based on the LCL assay. The gene expression profile 110GEP (e.g., transcriptome) may include a plurality of gene expression levels 110RSL, each representing quantity of a respective RNA molecule (e.g., sequenced RNA molecule 110RS) in LCL assay 20L.

As shown in steps S1015, processor 2 may provide a first ML based model 130, that may be pretrained to predict disposition of a mental disorder based, at least in part, on gene expression profile 110GEP data.

During an inference period, which may be subsequent to, or intermittent with training of ML model 130, processor 2 may apply ML-based model 130 on the gene expression profile 110GEP (or a subset 125SB thereof) of the subject, to predict disposition of the mental disorder (e.g., BD) in the subject.

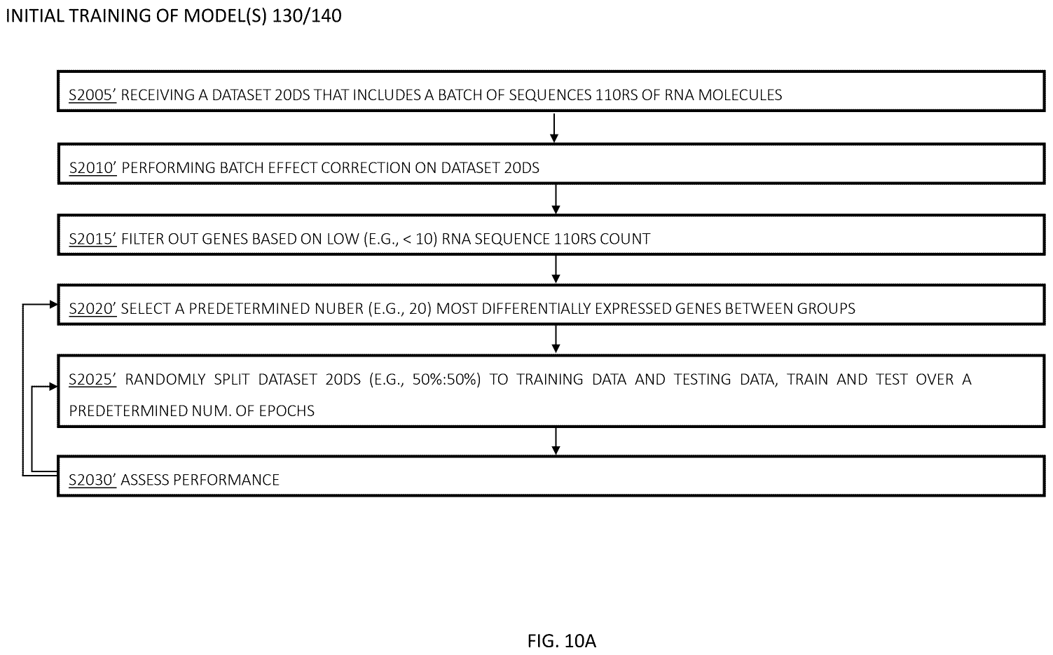

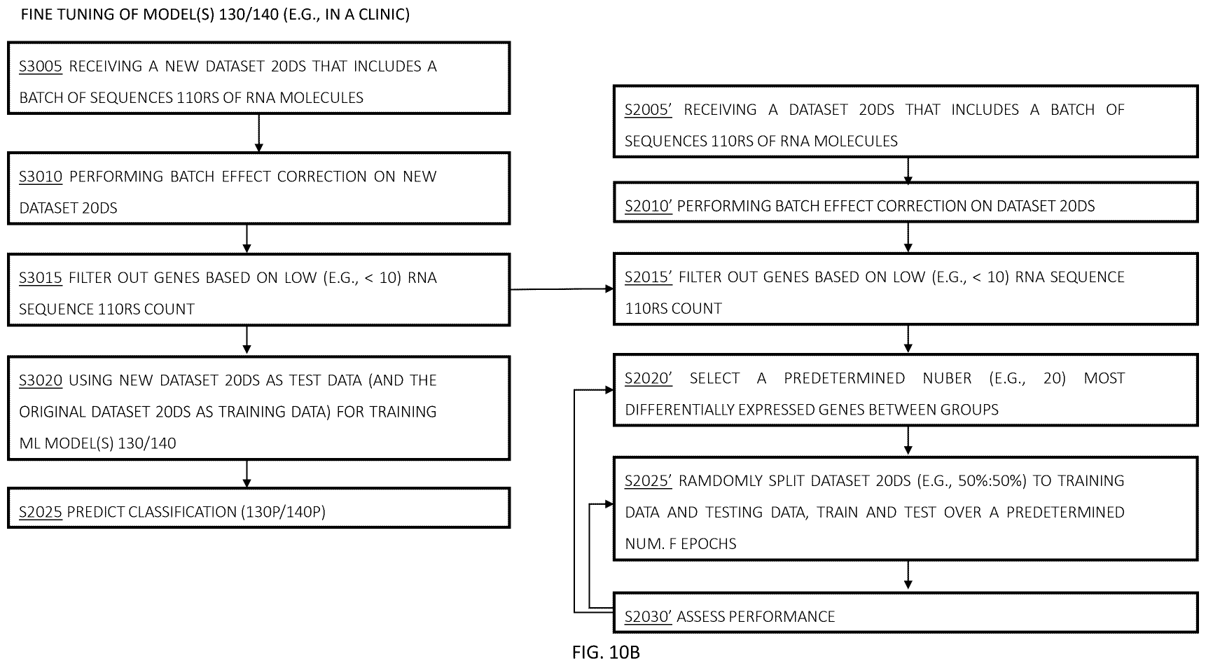

Reference is now made to FIGS. 10A and 10B, which are flow diagrams depicting a process of training ML model 130 and/or ML model 140, by training module 150, so as to predict (e.g., produce a prediction 130P) the condition of a subject (e.g., BD disposition), and/or predict (e.g., produce a prediction 140P) the patient's responsivity (e.g., LR/NR) to treatment, according to some embodiments of the invention.

FIG. 10A describes a process that may be employed during initial training of ML models 130/140.

As shown in step S2005, a processor (e.g., processor 2 of FIG. 1) of system 10 may receive a dataset 20DS (e.g., an annotated dataset 20DS) that includes a batch of sequences 110RS of RNA molecules.

As known in the art, batch effects are phenomena that arise from differences between samples that are not rooted in the experimental design and can have various sources, spanning from different handlers or experiment locations to different batches of reagents and even biological artifacts such as growth location. As shown in step S2010, processor 2 may utilize training module 150 to perform batch effect correction on dataset 20DS, based on any appropriate batch effect correction algorithm as known in the art.

As shown in step S2015, training module 150 may filter out, or omit genes (e.g., sequences 110RS) based on low (e.g., <10) RNA sequence 110RS count.

As shown in step S2020, training module 150 may select a predetermined number (e.g., 20) of the most differentially expressed genes between the relevant groups.

For example, when training ML model 130 to produce a prediction 130P of BD disposition of a subject, training module 150 may select a predetermined number of the most differentially expressed genes between BD and non-BD subjects, as depicted in the example of FIG. 3.

In another example, when training ML model 140 to produce a prediction 140P of Lithium responsivity (LR/NR) of a subject, training module 150 may select a predetermined number of the most differentially expressed genes between LR and NR patient, as depicted in the example of FIG. 6.

As shown in step S2025, training module 150 may randomly split dataset 20DS (e.g., 50%:50%) to a first group of training data sequences 110RS and a second group of testing data sequences 110RS. Training module 150 may subsequently train, and test the relevant ML model 130/140 over a predetermined number of epochs, and repeat the selection, training and testing, e.g., for a predetermined number of times.

As shown in step S2030, training module 150 may infer the relevant ML model 130/140, e.g., on one or more training data sequences 110RS, to assess metrics of performance (e.g., ROC, precision, recall, and the like) of the relevant ML model 130/140. According to some embodiments, when the metrics of performance are unsatisfactory (e.g., beneath a predefined threshold), training module 150 may proceed to retrain the relevant ML model 130/140 (e.g., return to steps S2020 or S2025), so as to improve the measured performance metrics.

FIG. 10B describes a process that may be employed during, or following deployment of system 10 (with initially trained ML models 130/140) e.g., in a clinic, to retrain, or refine a training of models 130/140 on patient-specific incoming data 20DS.

Note that steps S2005-S2030 in FIG. 10A may be substantially equivalent to respective steps S2005′-S2030′ in FIG. 10B, and their description will not be repeated, for the purpose of brevity.

As shown in step S3005, system 10 may receive a new dataset 20DS (e.g., an annotated dataset 20DS) that includes a batch of sequences 110RS of RNA molecules, pertaining to specific subjects in the environment (e.g., clinic) where system 10 is deployed.

As shown in step S3010, a processor (e.g., processor 2 of FIG. 1) may utilize training module 150 to perform batch-effect correction on the new dataset 20DS.

As shown in step S3015, training module 150 may filter out, or omit genes (e.g., sequences 110RS) of the new dataset 20DS, based on low (e.g., <10) RNA sequence 110RS count. At this stage, training module 150 may turn to step S2015′ (equivalent to S2015 of FIG. 10A), to complete the training process.

Additionally, or alternatively, training module 150 may turn to step S3020, where the new dataset 20DS may be used as test data, and the original dataset 20DS may be used as training data, for training ML model(s) 130/140.

As shown in step S3025, processor 2 may then employ ML model(s) 130/140, and infer them on incoming RNA sequence 110RS data, to predict a classification 130P/140P.

Embodiments of the invention may provide a practical application in the technological field of assistive diagnostics. Embodiments of the invention may include an improvement in this technology, by identifying a subset of differentially expressed genes indicative of BD disposition, and Lithium responsiveness.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of predicting disposition of a mental disorder of a subject by at least one processor, the method comprising:
obtaining a Lymphoblastoid Cell Line (LCL) assay of the subject;
calculating a gene expression profile of the subject based on the LCL assay, wherein said gene expression profile comprises a plurality of gene expression levels, each representing quantity of a respective RNA molecule in the LCL assay;
providing a first machine-learning (ML) based model, pretrained to predict disposition of a mental disorder based, at least in part, on gene expression profile data; and
applying the first ML-based model on the gene expression profile of the subject, to predict disposition of the mental disorder in the subject.

2. The method of claim 1, wherein the mental disorder is selected from a Bipolar Disorder (BD), a manic condition, and a condition of depression.

3. The method of claim 2, further comprising identifying, in the plurality of RNA molecules, a first subset of RNA molecules as differentially expressed between a first group of subjects, having the mental disorder, and a second, control group of subjects, beyond a predefined threshold,
and wherein applying the first ML-based model on the gene expression profile comprises applying the first ML-based model on the gene expression levels of the first subset of RNA molecules.

4. The method of claim 3, wherein the first subset of RNA molecules respectively correspond to a group of genes selected from: UBAP1L, OAZ3, RPL7P6, MTND5P15 and IGSF9T.

5. The method of claim 4, wherein the first subset of RNA molecules respectively correspond to a group of genes further selected from MYO1H, RPL29P33, OAZ3, RPL7P6, PPP1R3F, IGSF9, MTND5P15, UBAP1L, NEK10, SRC, PCDHGB7, SNORA20, DCBLD2, MRM2, TSACC, PPFIA1, ZC3H14, CHRM5, FRG1CP, and ZNF346.

6. The method of claim 1, further comprising:
obtaining clinical data representing historical manifestations of the mental disorder in the subject; and
applying the first ML-based model on the clinical data, in addition to the gene expression profile of the subject, to predict disposition of the mental disorder in the subject.

7. The method of claim 1, further comprising:
providing a second ML based model, pretrained to predict responsiveness to a treatment associated with the mental disorder based, at least in part, on gene expression profile data; and
applying the second ML-based model on the gene expression profile of the subject, to predict responsiveness of the subject to the treatment.

8. The method of claim 7, wherein the mental disorder is selected from a Bipolar Disorder (BD), a manic condition, and a condition of depression, and wherein the treatment comprises intake of Lithium.

9. The method of claim 8, further comprising identifying, in the plurality of RNA molecules, a second subset of RNA molecules as differentially expressed between a first group of subjects, responsive to the treatment, and a second group of subjects, not responsive to the treatment, beyond a predefined threshold,
and wherein applying the second ML-based model on the gene expression profile comprises applying the second ML-based model on the gene expression levels of the second subset of RNA molecules.

10. The method of claim 9, wherein the second subset of RNA molecules respectively correspond to a group of genes selected from: EEF1A1P34, NRIP2, GPR63, ADAM20P1, GLRA2, HCP5B and TERB1.

11. The method of claim 9, wherein the second subset of RNA molecules respectively correspond to a group of genes selected from: EEF1A1P34, NRIP2, GPR63, ADAM20P1, GLRA2, HCP5B, TERB1, SCAT2, NUSAP1, ZNF93, C16orf96, SNORA20, GPX2, IGHV5-51, CRYZ, WDR5-DT, IGLV1-47 and IGHV4-80.

12. The method of claim 9, further comprising:
obtaining clinical data representing historical manifestations of the mental disorder in the subject; and
applying the second ML-based model on the clinical data, in addition to the gene expression profile of the subject, to predict responsiveness of the subject to the treatment.

13. A system for predicting disposition of a mental disorder of a subject, the system comprising: a non-transitory memory device, wherein modules of instruction code are stored, and at least one processor associated with the memory device, and configured to execute the modules of instruction code, whereupon execution of said modules of instruction code, the at least one processor is configured to:
obtain a Lymphoblastoid Cell Line (LCL) assay of the subject;
calculate a gene expression profile of the subject based on the LCL assay, wherein said gene expression profile comprises a plurality of gene expression levels, each representing quantity of a respective RNA molecule in the LCL assay;
provide a first machine-learning (ML) based model, pretrained to predict disposition of a mental disorder based, at least in part, on gene expression profile data; and
apply the first ML-based model on the gene expression profile of the subject, to predict disposition of the mental disorder in the subject.

14. The system of claim 13, wherein the mental disorder is selected from a Bipolar Disorder (BD), a manic condition, and a condition of depression.

15. The system of claim 14, wherein the at least one processor is configured to:
identify, in the plurality of RNA molecules, a first subset of RNA molecules as differentially expressed between a first group of subjects, having the mental disorder, and a second, control group of subjects, beyond a predefined threshold; and
apply the first ML-based model on the gene expression profile by applying the first ML-based model on the gene expression levels of the first subset of RNA molecules.

16. (canceled)

17. The system of claim 15, wherein the first subset of RNA molecules respectively correspond to a group of genes further selected from UBAP1L, OAZ3, RPL7P6, MTND5P15, IGSF9T MYO1H, RPL29P33, OAZ3, RPL7P6, PPP1R3F, IGSF9, MTND5P15, UBAP1L, NEK10, SRC, PCDHGB7, SNORA20, DCBLD2, MRM2, TSACC, PPFIA1, ZC3H14, CHRM5, FRG1CP, and ZNF346.

18. The system of claim 13, wherein the at least one processor is further configured to:
obtain clinical data representing historical manifestations of the mental disorder in the subject; and
apply the first ML-based model on the clinical data, in addition to the gene expression profile of the subject, to predict disposition of the mental disorder in the subject.

19. The system of claim 13, wherein the at least one processor is further configured to:
providing a second ML based model, pretrained to predict responsiveness to a treatment associated with the mental disorder based, at least in part, on gene expression profile data; and
applying the second ML-based model on the gene expression profile of the subject, to predict responsiveness of the subject to the treatment.

20. The system of claim 19, wherein the mental disorder is selected from a Bipolar Disorder (BD), a manic condition, and a condition of depression, and wherein the treatment comprises intake of Lithium.

21. The system of claim 20, wherein the at least one processor is further configured to:
identify, in the plurality of RNA molecules, a second subset of RNA molecules as differentially expressed between a first group of subjects, responsive to the treatment, and a second group of subjects, not responsive to the treatment, beyond a predefined threshold; and
apply the second ML-based model on the gene expression profile by applying the second ML-based model on the gene expression levels of the second subset of RNA molecules.

22. (canceled)

23. (canceled)

24. (canceled)
